SEQUENCE LISTING (4 sequences)

SEQ ID NO: 1 - DNA, 21 bp, Artificial Sequence
  Synthetically generated oligonucleotide
agauguuccgcgagguugann

SEQ ID NO: 2 - DNA, 21 bp, Artificial Sequence
  Synthetically generated oligonucleotide
ucaaccucgcggaacaucunn

SEQ ID NO: 3 - DNA, 21 bp, Artificial Sequence
  Synthetically generated oligonucleotide
accgagaaauucacaagaunn

SEQ ID NO: 4 - DNA, 21 bp, Artificial Sequence
  Synthetically generated oligonucleotide
aucuugugaauuucucggunn

(19) United States
(12) Patent Application Publication
(10) Pub. No.: US 2009/0069231 A1
(43) Pub. Date: Mar. 12, 2009

(54) COMPOSITIONS AND METHODS FOR TREATING DIABETIC EYE DISEASE

(76) Inventors: Lloyd P. Aiello, Belmont, MA (US); Edward P. Feener, North Reading, MA (US)

Correspondence Address: FISH & RICHARDSON PC, P.O. BOX 1022, MINNEAPOLIS, MN, 55440-1022, US

(21) Appl. No.: 12/033,710

(22) Filed: Feb. 19, 2008

Related U.S. Application Data
(60) Provisional application No. 60/901,859, filed on Feb. 16, 2007.

(30) Foreign Application Priority Data
Feb. 16, 2006 (US) PCT/US2006/005395

(51) Int. Cl. A61K 38/16; G01N 33/53; C12Q 1/68; A61P 27/02

(52) U.S. Cl. 514/12; 435/7.1; 435/6

(57) ABSTRACT

The present invention provides methods and composition for the treatment and diagnosis of disorders associated with excessive retinal vascularisation and vascular permeability, e.g., diabetic retinopathy and diabetic macular edema.

### CLAIM OF PRIORITY

This application claims the benefit under 35 USC § 119(e) to U.S. Provisional Patent Application Ser. No. 60/901,859, filed on Feb. 16, 2007, the entire contents of which are hereby incorporated by reference.

### FEDERALLY SPONSORED RESEARCH OR DEVELOPMENT

This invention was made with U.S. Government support under Grant Nos. RO1-EY11289-16, R01-EY13178, P30-EY13078, EY014106, DK 60165 and DK 36836, awarded by the National Institutes of Health. The U.S. Government has certain rights in the invention.

### RELATED APPLICATIONS

This application is related to International Patent Application No. PCT/US2006/005395, filed on Feb. 16, 2006, and U.S. Provisional Patent Application Ser. Nos. 60/656,167, filed on Feb. 24, 2005, and 60/725,820, filed on Oct. 12, 2005. The entire contents of the foregoing applications are hereby incorporated by reference.

### TECHNICAL FIELD

This invention relates to compounds and methods for diagnosing and treating disorders associated with excessive vascular permeability and edema, e.g., in the retina and brain.

### BACKGROUND

Diabetic retinopathy (DR) is the leading cause of vision loss in working adults (Klein et al., Opthalmology 105:1801-1815 (1998); Ciulla et al., Diabetes Care 26:2653-2664 (2003)). Although its incidence and progression can be reduced by intensive glycemic and blood pressure control (The Diabetes Control and Complications Trial Research Group, N. Engl. J. Med. 329:977-986 (1993); Stratton et al., BMJ 321:405-412 (2000); UK Prospective Diabetes Study Group BMJ. 317:703-713 (1998) [published erratum appears in BMJ 1999 Jan. 2; 318(7175):29]), nearly all patients with type 1 diabetes mellitus (DM) and over 60% of those with type 2 DM develop retinal microvascular abnormalities termed nonproliferative diabetic retinopathy (NPDR), and 20% to 30% of these patients advance to active proliferative diabetic retinopathy (PDR) and/or diabetic macular edema (DME) (Aiello et al., Diabetes Care 21:143-156 (1998); Klein et al., Opthalmology 91:1464-1474 (1984); Javitt et al. Diabetes Care 17:909-917 (1994); Williams et al., Eye 18:963-983 (2004)). While photocoagulation surgery and vitrectomy are highly effective in reducing vision loss, preventative treatments for PDR and DME remain a major unmet clinical need.

Increased retinal vascularisation and retinal vascular permeability (RVP) are primary causes of DME and a characteristic finding in PDR, as well as other disorders.

### SUMMARY

The present invention is based, at least in part, on the identification of Dickkopf 3 (DKK3) as an endogenous inhibitor of angiogenesis and permeability in the eye. DKK3 concentrations are reduced in the eye of subjects with diabetes, reducing the inhibition of angiogenesis and permeability, leading to complications. Therefore, enhancing DKK3 levels or activity provides new therapeutic opportunities in the treatment of diabetic retinopathy, e.g., proliferative diabetic retinopathy, and diabetic macular edema. The proteins identified in the screens described herein are useful in novel therapeutic and diagnostic methods for treating diabetic retinopathy, e.g., proliferative diabetic retinopathy, and diabetic macular edema.

In one aspect, the invention provides methods for the treatment of disorders associated with excessive vascular permeability or vascularisation of the eye, e.g., diabetic retinopathy, e.g., proliferative diabetic retinopathy, and diabetic macular edema. The methods include administering to the subject a therapeutically effective amount of one or more of a composition that increases activity or levels of DKK3, e.g., a DKK3 polypeptide or active fragment thereof, or a nucleic acid encoding DKK3 or an active fragment thereof.

The methods can include administering a composition described herein by local administration to the eye of the subject, e.g., by injection into or near the eye, e.g., into the vitreous or aqueous humor of the eye, or by intrabulbar injection, or by administration as eye drops. In some embodiments, the methods include the use of a local drug delivery device (e.g., a pump or a biocompatible matrix) to deliver the composition. In other embodiments, the composition is delivered via injection into the cerebral fluid or cerebral spinal fluid. In some embodiments, the administration is systemic.

As used herein, disorders associated with excessive vascular permeability or vascularisation of the eye include diabetic retinopathy, diabetic macular edema, and proliferative diabetic retinopathy. Described herein are methods of treating such disorders, e.g., by decreasing vascular permeability or vascularisation, or preventing future increases in vascular permeability or vascularisation in those subjects identified as at risk of developing excessive vascular permeability or vascularisation, e.g., subjects with early stages of DR. In some embodiments, the methods described include a step of selecting a subject on the basis that the subject has, or is at risk for developing, a disorder associated with excessive vascular permeability or vascularisation of the eye, as described herein.

In some embodiments, the disorder associated with increased vascular permeability or vascularisation is also associated with hemorrhage, i.e., bleeding into the affected area. In some embodiments, the disorder associated with increased vascular permeability or permeability is also associated with lysis of erythrocytes in the affected area.

The invention also includes the pharmaceutical compositions described herein, e.g., compositions including a compound that increases levels or activity of DKK3, and a physiologically acceptable carrier. In some embodiments, the composition is adapted for injection into the vitreous or aqueous humor of a mammalian eye, or for use as eye drops. In some embodiments, the composition is adapted for intrathecal, e.g., subdural or subarachnoid, delivery.

The methods described herein also include methods for diagnosing a subject with a disorder associated with excessive vascular permeability or vascularisation as described herein, by detecting a level of DKK3 in a sample from the subject, e.g., from the eye (e.g., the vitreous or aqueous humor) of the subject and comparing the level to a reference, e.g., a control reference that represents the level of DKK3 in an unaffected subject. The presence of a level of DKK3 that is reduced, i.e., significantly decreased, as compared to the reference indicates that the subject has a disorder associated with excessive vascular permeability or vascularisation.

In another aspect, the invention includes methods for selecting a subject or population of subjects for participation in a clinical trial of a treatment for a disorder associated with excessive vascularisation or vascular permeability. The methods include detecting a level of DKK3 in a sample from the subject, e.g., from the eye (e.g., the vitreous or aqueous humor) of the subject; and selecting the subject on the basis of the level of DKK3, optionally as compared to a reference, e.g. a preselected threshold below or above which the subject is selected. For example, the subjects can be selected for or against if they have a subnormal level of DKK3, or can be categorized depending on the level of DKK3.

In some embodiments, the invention includes methods for identifying a candidate compound for the treatment of a disorder associated with excessive vascularisation or vascular permeability of the eye. The methods include assaying DKK3 levels or activity in the presence of a test compound. Compounds that increase DKK3 levels or activity, e.g., relative to a reference, can then be selected as candidate compounds for the treatment of the disorder.

The invention further provides methods for identifying candidate compounds for the treatment of a disorder associated with excessive vascular permeability or vascularisation in a model. The methods include providing a model (e.g., an animal model) of a disorder associated with excessive vascular permeability or vascularisation of the eye, e.g., diabetic retinopathy, e.g., proliferative diabetic retinopathy, and diabetic macular edema; contacting the model (e.g., an animal of an animal model) with a test compound; detecting a level of DKK3 protein in the model, or mRNA encoding DKK3; and comparing the level of the DKK3 protein or mRNA to a reference, e.g., a control reference that represents the level of DKK3 in an untreated model. A test compound that causes a significant increase in a level of the DKK3 protein or mRNA, e.g., as compared to the reference, is a candidate compound that may be potentially useful for the treatment of a disorder associated with excessive vascular permeability or vascularisation of the eye, e.g., diabetic retinopathy, e.g., proliferative diabetic retinopathy, or diabetic macular edema.

In some embodiments, the invention includes methods for diagnosing a disorder associated with excessive retinal vascular permeability of the eye in a subject. Such methods include obtaining a test sample from the subject and detecting a level of DKK3 protein or mRNA encoding DKK3 in the sample. The level of DKK3 in the test sample can then be compared to the level of DKK3 protein or mRNA, e.g., in a reference sample. A decrease in a level of the DKK3 protein or mRNA in the test sample as compared to the reference sample can indicate that the subject has a disorder associated with excessive retinal vascularisation or vascular permeability.

The invention also includes methods for evaluating a treatment for a disorder associated with excessive vascular permeability or vascularisation of the eye, e.g., diabetic retinopathy, diabetic macular edema, and proliferative diabetic retinopathy. The methods include detecting a level of DKK3 protein or mRNA encoding DKK3 in a sample from a subject, e.g., from the eye, e.g., the vitreous or aqueous humor, of the subject; administering one or more doses of a treatment to the subject, and comparing the level of DKK3 protein or mRNA to a reference, e.g., a level of the protein or mRNA prior to administration of the treatment. A significant increase in a level of the protein or mRNA as compared to the reference indicates that the treatment is effective.

The invention additionally provides methods for determining a subject's risk for developing a sight-threatening complication of retinopathy. Such methods include detecting a level of DKK3 protein or mRNA encoding DKK3 in a sample from the subject, and comparing the level of the DKK3 protein or mRNA to a reference. A difference in the level of the DKK3 protein or mRNA as compared to the reference indicates the subject's risk of developing a sight-threatening complication of retinopathy.

The invention also includes methods for determining when a treatment modality (e.g., administration of a compound as described herein, or another method of treating a disorder associated with excessive vascular permeability or vascularisation of the eye, e.g., diabetic retinopathy, diabetic macular edema, and proliferative diabetic retinopathy) that is administered to a subject to treat or prevent a disorder associated with excessive vascular permeability can be stopped or altered, e.g., reduced to a maintenance phase. The methods include providing a sample from the subject, e.g., from the eye (e.g., the vitreous or aqueous humor) of the subject; detecting a level of DKK3 protein or mRNA encoding DKK3 in the sample; comparing the level of DKK3 protein or mRNA to a reference, e.g., a level of the protein or mRNA in an unaffected subject. A level of DKK3 protein or mRNA that approaches (e.g., is not significantly different from) the level of DKK3 protein or mRNA in a normal subject indicates that the treatment can be stopped or reduced to a maintenance phase.

Further, the invention includes methods for determining when a treatment for a disorder associated with excessive vascular permeability or vascularisation should be initiated in a subject. The methods include providing a sample from the subject, e.g., from the eye, e.g., the vitreous or aqueous humor, of the subject; detecting a level of DKK3 protein or mRNA encoding DKK3 in the sample; and comparing the level of DKK3 protein or mRNA to a reference. A decrease, e.g., a significant decrease, in a level of DKK3 protein or mRNA, e.g., as compared to the reference, indicates that the treatment should be initiated.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications including U.S. Ser. No. 60/901,589, filed on Feb. 16, 2007, patents, reference to public sequence database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a photograph of a Northern blot of DKK3 performed in retinal pericytes (RPEs), retinal endothelial cells (RECs), and retinal pigment cells (RPCs). 36B4 is a loading control.

FIG. 1B is a bar graph showing mean data regarding relative DKK3 expression from the Northern blot data shown in FIG. 1A.

FIG. 2A is a photograph of a Northern blot showing the effect of serum (calf serum (CS)) on DKK3 expression in RPCs. DKK3 expression is shown in the top panel. The loading control is shown in the bottom panel. Serum concentrations were 0%, 0.1%, 2%, 10%, and 20% calf serum (CS).

FIG. 2B is a bar graph showing quantification of the Northern blot data shown in FIG. 2A.

FIG. 3A shows the effect of insulin on DKK3 expression over time. The top two panels show photographs of Northern blots from two independent experiments. DKK3 expression is shown in the top row. 36B4 is shown as a loading control in the bottom row. 1, 4, 10, and 24 indicate the time point following the addition of insulin at which samples were taken Mean values from each of these experiments are shown in the form of a bar graph in the bottom panel.

FIG. 3B shows the effect of high glucose concentration on DKK3 expression. The top panel shows a Northern blot of DKK3 expression and 36B4 expression (as a loading control). Mean data from three independent experiments is shown as a histogram in the bottom panel.

FIGS. 4A-4C show the effect of insulin on DKK3 expression. FIGS. 4A and 4B are photographs of Western blots showing DKK3 expression at 24 and 36 hours after stimulation with 100 nM insulin. FIG. 4A shows DKK3 expression in the cells. FIG. 4B shows DKK3 expression in the media (e.g., secreted DKK3). FIG. 4C shows mean data from FIG. 4A and FIG. 4B as a bar graph.

FIG. 5 is a set of photographs of Western blots performed to measure DKK3 expression levels in vitreous. The left panel shows DKK3 expression in a Bovine sample. The right panel shows DKK3 expression in a rat sample.

FIG. 6A is a Western blot showing DKK3 levels in non-DM subjects and subject with PDR.

FIG. 6B is a Western blot showing DKK3 expression levels in patient vitreous. Samples represent macular hole (MH) and retinal detachment (RD).

FIG. 7A is a set of two bar graphs depicting levels of DKK3 expression in the brain (upper graph) and heart (lower graph) of streptozotocin (STZ)-induced rat model of diabetes (DM) and untreated control animals (control) two months post treatment. Data is representative of four independent experiments.

FIG. 7B is a bar graph showing DKK3 expression in rat retinas two months after STZ treatment.

FIG. 8A is a photograph showing the morphology and size of RPE cells treated with siRNA against GFP (left side) and DKK3 (right side) 2 days after transfection. Cells were treated with siRNA at a final concentration of 100 nM. siRNA treated cells are smaller than GFP treated cells.

FIG. 8B is a graph showing relative DKK3 expression levels following siRNA treatment as assessed using real-time PCR (RT PCR).

FIG. 8C is a bar graph showing relative DKK3 expression levels following siRNA treatment, as assessed by semi-quantitative RT PCR of the cells.

FIG. 9A is a photograph showing the morphology and size of bovine AoECs treated with siRNA against GFP and DKK3. DKK3 siRNA treated cells were slow growing and did not reach confluency.

FIG. 9B is a bar graph showing cell numbers in samples treated with siRNA against GFP and DKK3.

FIG. 9C is a bar graph showing DKK3 expression in cells treated with siRNA against DKK3.

FIG. 9D is a Western blot (top panel) and bar graph (bottom panel) showing the effects of the treatment of cells with siRNA against DKK3 on DKK3 protein expression levels.

FIG. 10A is a set of bar graphs showing endothelial cell growth. Top panel-* is p=<0.05. Bottom panel-* is p=<0.05 versus control. # is p=<0.05 versus VEGF.

FIG. 10B is a set of bar graphs showing endothelial migration. Top panel-* is p<0.05; # is p=<0.01. P values were determined using the Student's T-test. Bottom panel-# is p<0.01 control versus VEGF determined using the Student's T-test. * is p=0.06 determined using analysis of variance (ANOVA; N=4). P=0.008 determined by ANOVA in VEGF stimulated groups (N=3).

FIG. 10C is a Western blot showing Erk phosphorylation in DKK3 treated AoECs. Cells were treated with 0.1% albumin overnight, followed by treatment with 10 μg/mL DKK3. Control cells were treated with 25 ng/mL VEGF for 15 minutes.

FIG. 10D top and middle panels are Western blots showing phosphorylated and total Erk in RECs treated with VEGF (V) or VEGF and DKK3 (V+D). Cells were cultured in 2% fetal bovine serum (FBS) overnight. Cells were then pretreated with DKK3 for 15 minutes prior to treatment with VEGF (25 mg/mL) for 10 minutes. The bottom panel is a bar graph showing the mean of three independent experiments.

FIG. 10E is an immunoblot showing VEGF-induced KDR phosphorylation. RECs were cultured in 2% FBS overnight. Cells were then pretreated with DKK3 for the indicated time, followed by stimulation with VEGF (25 ng/mL) (“V+D”) for 5 minutes. “C” depicts control cells. “V” depicts cells treated with VEGF (without DKK3). Phosphorylated KDR was immunoprecipitated using an anti-KDR antibody and membranes were probed using an anti-phosphotyrosine antibody.

FIG. 11A is a bar graph showing the results of a functional ELISA performed to evaluate the interaction between VEGF and DKK3 in samples incubated with DKK3 (1000 ng/ml; left bar of each pair of bars), or albumin (1000 ng/ml; right bar of each pair of bars). Data presented in the chart is representative of four independent experiments (n=4).

FIG. 11B is two bar graphs showing the interaction between DKK3 and VEGF as assessed using scintillation. The left panel shows the counts per minute (cpm) for each of the three experimental conditions. The right panel shows this data expressed as a percentage of the control (DKK3 antibody) (N=1).

FIG. 12 is a set of bar graphs showing the inhibitory effect of DKK3 on the interaction between VEGF and the VEGF receptor (VEGFR). Data are representative of two independent experiments.

FIG. 13 is a bar graph showing the effect of DKK3 on retinal vascular permeability. P values are shown in the legend.

FIG. 14A is a bar graph showing endothelial cell viability in the presence of 10% calf serum (the left bar of each pair of bars) and 0.1% serum (the right bar of each pair of bars). 5, 50, and 500 indicate the concentration of DKK3 present in nM. Cell viability in the presence of albumin was 1. * indicates a statistically significant difference. P=0.09. Data are representative of four independent experiments.

FIG. 14B is a line graph showing cell viability in REC and HUVEC cells both in the presence of 10% and 0.1% serum. 1, 10, 100, and 1000 indicate the concentration of DKK3 present in nM. Data are representative of four independent experiments. **=p<0.01. REC 0.1% serum p=0.01; HUVEC 10% serum p=0.025; REC 10% serum p=0.09.

### DETAILED DESCRIPTION

The onset of proliferative diabetic retinopathy and diabetic macular edema is thought to result from a change in the balance between pro-angiogenic/permeability factors and anti-angiogenic/permeability factors. Functional proteomics was used to identify novel factor(s) that contribute to the changes observed in the eye associated with diabetes. Such changes include excessive vascular permeability or vascularisation of the eye, e.g., associated with diabetic retinopathy, e.g., proliferative diabetic retinopathy, and diabetic macular edema. The effect on the vitreous proteome was characterized using mass spectrometry to inventory and compare the vitreous protein composition of nondiabetic subjects and diabetic patients with or without active PDR and macular edema.

As previously described in International Patent Application No. PCT/US2006/005395, filed Feb. 16, 2006, these studies identified a number of proteins whose expression levels are altered in PDR and/or macular edema. Among those proteins is Dickkopf 3 (DKK3).

As described herein, DKK3 concentrations are reduced with diabetes, reducing the inhibition of angiogenesis and permeability and thus allowing complications from these to arise. DKK3 exerts its action in part by interaction with the VEGF receptor. Thus, methods are described herein for identifying compounds that interact with the VEGF receptor in a way similar to DKK3. Such compounds are expected to have therapeutic potential for the treatment of diabetic retinopathy.

DKK3 has tumor suppressor, apoptosis and anti-proliferative activities and is involved in cellular migration and differentiation. The expression and regulation of DKK3 was evaluated in retina and cultured retinal cells. DKK3 mRNA expression in bovine retinal capillary endothelial cells (REC), pericytes (RPC) and pigment epithelial cells (RPE) were assessed by Northern blot and RT-PCR analysis. Retinal DKK3 expression was evaluated in control and streptozotocin-induced diabetic mice (C57/BL6) by RT-PCR.

DKK3 gene expression was detected in all cultured retinal cells examined. Expression was 10.3- and 3.8-fold higher in RPC and RPE, respectively, than in REC. DKK3 expression was increased by cellular confluency 1.4- and 2.3-fold in REC and RPE, respectively. High glucose exposure (22 mM) for 5 days decreased DKK3 gene expression in RPC 24% (p=0.039) as compared with normal glucose (5.5 mM) of equal osmolarity. Insulin (100 nM) increased DKK3 gene expression in RPC in a time and dose-dependent manner (180%+30% at 4 hours), an effect maintained at least 10 hours. DKK2 and DKK3, but not DKK1, were detected in normal mouse retina. DKK3 gene expression decreased in mouse retina after 8 weeks of diabetes by 24% (p=0.017) as compared to nondiabetic retina.

Thus, DKK3 expression is decreased in diabetic retina and is regulated by cell confluency, glucose concentration, and insulin in retinal cells in culture. These findings provide evidence for a regulatory role of DKK3 in mediating retinal complications of diabetes.

Dickkopf 3 (DKK3)

The DKK family includes four isoforms. Except DKK3, all of the DKK family members (i.e., DKK1, DKK2, and DKK4) can bind Wnt receptors, resulting in blocking the beta-catenin pathway. It was previously known that DKK3 does not bind Wnt receptors, but does inhibit the beta-catenin signalling pathway, but the mechanism by which this occurs was not clear. DKK3 is also known as REIC. The human DKK3 mRNA has three variants, as follows, all of which encode the same protein:
Variant 1 represents the longest transcript; see Nucleic Acid Sequence Genbank Acc No.: NM_{—}001018057.1, Amino Acid Sequence Genbank Acc No.: NP_{—}001018067.1.
Variant 2 differs in the 5′ UTR compared to variant 1; see Nucleic Acid sequence Genbank Acc No.: NM_{—}013253.4, Amino Acid sequence Genbank Acc No.: NP_{—}037385.2.
Variant 3 differs in the 5′ UTR compared to variant 1; see Nucleic Acid Genbank Acc No.: NM_{—}001018057.1; Amino Acid GenbankAcc No.: NP_{—}001018067.1.

The human DKK3 protein contains two conserved domains. The first, at amino acids 146-197, is known as the Dickkopf N-terminal cysteine-rich region. The second is at amino acids 207-273, and is known is a Prokineticin domain.

Pharmaceutical Compositions and Methods of Administration

Described herein are pharmaceutical compositions that include as active agents an enhancer of DKK3 levels or activity. The methods described herein include the manufacture and use of such pharmaceutical compositions.

In some embodiments, the invention includes a pharmaceutical composition that includes a DKK3 polypeptide or active fragment thereof, or a nucleic acid encoding a DKK3 polypeptide or active fragment thereof.

Formulation of Pharmaceutical Compositions

Pharmaceutical compositions typically include the active ingredient and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is typically formulated to be compatible with its intended route of administration. Examples of routes of administration include parenteral, e.g., intravenous, intradermal, subcutaneous, oral (e.g., inhalation), intrathecal (e.g., subdural or subarachnoid), transdermal (topical), transmucosal, and rectal administration. In some embodiments, e.g., for treating disorders associated with excessive retinal vascular permeability, the composition is administered directly to the eye, e.g., by eye drops, or directly into the eye across the blood-retinal barrier, e.g., by implants, peribulbar injection, or intravitreous injection. In some embodiments, e.g., for treating disorders associated with excessive cerebral vascular permeability, the composition is delivered across the blood-brain barrier, e.g., intrathecal, e.g., subdural or subarachnoid delivery, e.g., delivery into the cerebral or cerebrospinal fluid. In some embodiments, e.g., for administration to the vitreous or retina, the active ingredient is incorporated into a polymer matrix that is implanted into or near the site of intended delivery.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol or sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the composition is especially adapted for administration into or around the eye. For example, a composition can be adapted to be used as eye drops, or injected into the eye, e.g., using peribulbar or intravitreal injection. Such compositions should be sterile and substantially endotoxin-free, and within an acceptable range of pH. Certain preservatives are thought not to be good for the eye, so that in some embodiments a non-preserved formulation is used. Formulation of eye medications is known in the art, see, e.g., Ocular Therapeutics and Drug Delivery: A Multi-Disciplinary Approach, Reddy, Ed. (CRC Press 1995); Kaur and Kanwar, Drug Dev Ind Pharm. 2002 May; 28(5):473-93; Clinical Ocular Pharmacology, Bartlett et al. (Butterworth-Heinemann; 4th edition (Mar. 15, 2001)); and Ophthalmic Drug Delivery Systems (Drugs and the Pharmaceutical Sciences: a Series of Textbooks and Monographs), Mitra (Marcel Dekker; 2nd Rev&Ex edition (Mar. 1, 2003)).

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Administration of a therapeutic compound described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Compositions including nucleic acid compounds can be administered by any method suitable for administration of nucleic acid agents. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996). In some embodiments, the nucleic acid compounds comprise naked DNA, and are administered directly, e.g., as described herein. The DKK3 nucleic acid molecules described herein can be administered to a subject (e.g., by direct injection at a tissue site). Alternatively, DKK3 nucleic acid molecules can be modified to target selected cells, e.g., retinal cells, and then administered systemically. For systemic administration, DKK3 nucleic acid molecules can be modified such that they specifically bind to receptors or antigens expressed on a selected cell surface, e.g., by linking the DKK3 nucleic acid nucleic acid molecules to peptides or antibodies that bind to cell surface receptors or antigens. The DKK3 nucleic acid molecules can also be delivered to cells using the vectors described herein. To achieve sufficient intracellular concentrations of the DKK3 nucleic acid molecules, vector constructs in which the DKK3 nucleic acid nucleic acid molecule is placed under the control of a strong promoter can be used.

In some embodiments, the compositions are prepared with carriers that will protect the active ingredient against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially, e.g., from Alza Corporation or Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The delivery systems can include time-release, delayed release or sustained release delivery systems. Such systems can avoid repeated administrations of the agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109.

Delivery systems can also include non-polymer systems, e.g., lipids including sterols such as cholesterol, cholesterol esters and fatty acids or neutral fats such as mono-, di- and tri-glycerides; hydrogel release systems; sylastic systems; peptide based systems; wax coatings; compressed tablets using conventional binders and excipients; partially fused implants; and the like. Specific examples include, but are not limited to erosional systems in which the active agent is contained in a form within a matrix such as those described in U.S. Pat. Nos. 4,452,775, 4,667,014, 4,748,034 and 5,239,660, and diffusional systems in which an active component permeates at a controlled rate from a polymer such as described in U.S. Pat. Nos. 3,832,253, and 3,854,480. Pump-based hardware delivery systems can be used, some of which are adapted for implantation. In addition, U.S. Pat. No. 6,331,313 describes a biocompatible ocular drug delivery implant device that can be used to deliver active agents directly to the macular region.

Use of a long-term sustained release implant may be particularly suitable for treatment of chronic conditions. Long-term release means that the implant is constructed and arranged to delivery therapeutic levels of the active ingredient for at least 30 days, e.g., 60 days. Long-term sustained release implants are known to those in the art and include some of the release systems described herein.

The pharmaceutical compositions can be included in a container, pack, or dispenser together with instructions for administration.

Methods of Treatment

The pharmaceutical compositions described herein are useful in the treatment of disorders associated with increased vascular permeability, as described herein.

As used in this context, to “treat” means to ameliorate at least one symptom of the disorder associated with increased vascularisation of the eye. Often, increased vascularisation results in reduced vision; thus, a treatment can result in a reduction in vascularisation and a return or approach to normal vision. Administration of a therapeutically effective amount of a composition described herein for the treatment of a condition associated with increased vascularisation will result in decreased vascularisation, or at least a cessation in neovascularization (the formation of new blood vessels). In diabetic retinopathy, macular edema, or proliferative diabetic retinopathy, administration of a therapeutically effective amount of a composition described herein may result in unobstructed vision, improved vision or reduction in the rate of visual loss.

Dosage, toxicity and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in a method described herein, the therapeutically effective dose can be estimated initially from animal studies, e.g., from intravitreal injection in animals. A dose may be formulated in animal models to achieve a circulating plasma concentration range that includes the IC50 (i.e., the concentration of the test compound which achieves a half-maximal inhibition of symptoms) as determined in intravitreal injection. Such information can be used to more accurately determine useful doses in humans. Levels in plasma or vitreous may be measured, for example, by high performance liquid chromatography and mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered, e.g., from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include, e.g., a single treatment or a series of treatments.

Nucleic Acid Therapy

The nucleic acids described herein, e.g., nucleic acids encoding DKK3 or an active fragment thereof, can be incorporated into gene constructs to be used as a part of a gene therapy protocol. The methods include the use of expression vectors for in vivo transfection and expression of DKK3 in the eye. Expression constructs can be administered in any biologically effective carrier, e.g. any formulation or composition capable of effectively delivering the component gene to cells, e.g., retinal cells, in vivo. Approaches include insertion of the sequence encoding DKK3 in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (LIPOFECTIN™) or derivatized (e.g. antibody conjugated), polylysine conjugates, gramicidin S, artificial viral envelopes, or other such intracellular carriers, as well as direct injection of the gene construct or CaPO₄ precipitation carried out in vivo.

An exemplary approach for in vivo introduction of nucleic acid into a cell is by use of a viral vector containing nucleic acid, e.g. a cDNA, encoding DKK3. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Current Protocols in Molecular Biology, Ausubel, et al. (eds.) Greene Publishing Associates, (1989), Sections 9.10-9.14 and other standard laboratory manuals. Examples of suitable retroviruses include pLJ, pZIP, pWL and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68: 143-155. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of DKK3 in the tissue of a subject. Most nonviral methods of gene transfer rely on normal mechanisms used by mammalian cells for the uptake and intracellular transport of macromolecules. In some embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

In a representative embodiment, a gene encoding DKK3 can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the delivery systems for the DKK3 nucleic acid can be introduced into a patient by any of a number of methods, each of which is known in the art. For instance, a pharmaceutical preparation of the nucleic acid delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant nucleic acid is more limited with introduction into the animal being quite localized. For example, the nucleic acid delivery vehicle can be introduced by catheter (see U.S. Pat. No. 5,328,470) or by stereotactic injection (e.g. Chen et al. (1994) PNAS 91: 3054-3057).

The pharmaceutical preparation of the nucleic acid construct can consist essentially of the nucleic acid delivery system in an acceptable diluent, or can comprise a slow release matrix in which the nucleic acid delivery vehicle is imbedded. Alternatively, where the complete nucleic acid delivery system can be produced in tact from recombinant cells, e.g. retroviral vectors, the pharmaceutical preparation can comprise one or more cells which produce the nucleic acid delivery system.

Methods of Diagnosis

Also described herein are methods for diagnosing a disorder associated with excessive vascularisation and permeability as described herein, e.g., a disorder associated with excessive retinal or vitreal vascularisation or vascular permeability. The methods include obtaining a sample from a subject, and evaluating the presence and/or level of DKK3 in the sample, and comparing the presence and/or level with one or more references, e.g., a control reference that represents a normal level of the proteins, and/or a disease reference that represents a level of the proteins associated with diabetic retinopathy or macular edema. Suitable reference values can include those shown in Table 1.

TABLE 1

| | Range | Median | Mean ± SD |
|---|---|---|---|
| All Subjects (n = 12) | 0-6 | 3.0 | 2.6 ± 2.2 |
| No DM Only (n = 5) | 2-5 | 4 | 3.6 ± 1.1 |
| DM Only (n = 4) | 0-6 | 2.0 | 2.5 ± 3.0 |
| PDR Only (n = 3) | 0-3 | 0.0 | 1.0 ± 1.7 |

Group Differences: Chi-Square = 2.77; P value = 0.25

The presence and/or level of DKK3 protein can be evaluated using methods known in the art, e.g., using quantitative immunoassay methods. In some embodiments, high throughput methods, e.g., protein or gene chips as are known in the art (see, e.g., Ch. 12, Genomics, in Griffiths et al., Eds. Modern Genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of DKK3 proteins.

In some embodiments, the presence and/or level of DKK3 protein is comparable to the presence and/or level of the protein(s) in the disease reference, and the subject has one or more symptoms associated with diabetic retinopathy or macular edema, then the subject has diabetic retinopathy. In some embodiments, the subject has no overt signs or symptoms of diabetic retinopathy or macular edema, but the presence and/or level of one or more of the proteins evaluated is comparable to the presence and/or level of the protein(s) in the disease reference, then the subject has an increased risk of developing diabetic retinopathy. In some embodiments, the sample includes vitreous fluid; in other embodiments, the sample includes aqueous fluid. In some embodiments, once it has been determined that a person has diabetic retinopathy, or has an increased risk of developing diabetic retinopathy, then a treatment as described herein can be administered.

Methods of Screening

The invention includes methods for screening test compounds, e.g., polypeptides, peptides, polynucleotides, inorganic or organic large or small molecule test compounds, to identify agents useful in the treatment of opthalmological disorders associated with increased vascularisation and vascular permeability, e.g., diabetic retinopathy or macular edema. The methods include the use of assays that identify test compounds that can increase activity or levels of DKK3 protein. In some embodiments, the methods include the use of assays that identify compounds that mimic the action of DKK3 on VEGF/VEGFR signaling.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The small molecules can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the small organic molecules and libraries thereof can be obtained by systematically altering the structure of a first small molecule, e.g., a first small molecule that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a cell or living tissue or organ, e.g., an eye, and one or more effects of the test compound is evaluated. In a cultured or primary cell for example, the ability of the test compound to increase expression of DKK3 can be evaluated. In the eye, for example, the ability of the test compounds to increase expression of DKK3 can be evaluated.

In some embodiments, the test sample is an “engineered” in vivo model. For example, vitreous from a human subject, e.g., a human subject having diabetic retinopathy, can be transplanted into one or both eyes of an animal model, e.g., a rodent such as a rat. For example, about 10 μl of human vitreous can be injected into the rat vitreous compartment and the response on retinal vascular permeability measured. Alternatively or in addition, purified DKK3 protein or nucleic acid can be injected. In some embodiments, the model animal also has diabetes, e.g., a streptozotocin-induced or genetic animal model of diabetes. In some experiments, the DKK3 or human vitreous will be co-injected with other agents, e.g., test compounds, such as known or potential enhancers of DKK3.

Methods for evaluating each of these effects are known in the art. For example, ability to modulate expression of DKK3 protein can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect an effect on DKK3 levels. Vascular permeability can be evaluated, e.g., as described in International Patent Application No. PCT/US2006/005395, filed on Feb. 16, 1006.

Test compounds identified as “hits” (e.g., test compounds that increase the levels and/or activity of DKK3 can be selected and systematically altered, e.g., using rational design, to optimize binding affinity, avidity, specificity, or other parameters. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating opthalmological disorders associated with increased retinal vascular permeability, as described herein, e.g., diabetic retinopathy. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of an opthalmological disorder associated with increased vascularisation and/or vascular permeability, as described herein. The animal can be monitored for a change in the disorder, e.g., for an improvement in a parameter of the disorder, e.g., a parameter related to clinical outcome. In some embodiments, the parameter is vascularisation or vascular permeability, and an improvement would be a decrease in vascularisation or vascular permeability. In some embodiments, the subject is a human, e.g., a human with diabetes, and the parameter is visual acuity.

The invention is further described in the following examples, which do not limit the scope of the invention described in the claims.

### EXAMPLES

### Example 1

### Protein Inventory of Human Vitreous

To catalog the proteins present in human vitreous, proteomic analysis was performed.

Vitreous fluid was obtained from patients undergoing pars plana vitrectomy at the Beth Israel Deaconess Medical Center (Boston, Mass.) and Santa Barbara Cottage Hospital Eye Center (Santa Barbara, Calif.) in accordance with institutional review boards at both institutions. The reasons for vitrectomy included macular hole, epiretinal membrane, glaucoma, and retinal detachment. Samples were stored at −80° C. until used.

Vitreous samples were obtained from pars plana vitrectomy of non-diabetic mellitus (NDM) subjects (n=9), patients with diabetes mellitus and no diabetic retinopathy (noDR, n=4), and diabetic patients with PDR+/−DME (PDR, n=12). The study subject demographics are summarized in Table 1. This study included 25 subjects, including 7 females and 18 males. The PDR group subjects were younger (P <0.05) than the NDM and noDR groups. Eight subjects in the PDR group were diagnosed with diabetic macular edema.

TABLE 1
Demographics of Study Subjects.

| Group and | | | | DM | | |
|---|---|---|---|---|---|---|
| Level of | | Age years | Gender | (type | Duration | DME |
| Retinopathy | n | (mean ± SD) | (F/M) | 1/2) | (mean ± SD) | (y/n) |
| NDM | 8 | 72.4 ± 7.8 | 3/5 | n/a | n/a | n/a |
| noDR | 4 | 74. ± 6.0 | 1/3 | 0/4 | 13.5 ± 15.6 | 0/4 |
| PDR | 13 | 49.9 ± 9.3* | 3/10 | 8/5 | 21.6 ± 8.4 | 8/5 |

Diabetic Macular Edema (DME)
*P < 0.001

Proteomic analysis was performed on 50 μL of undiluted vitreous from non-diabetic mellitus (NDM) subject (n=5), patients with diabetes mellitus with no diabetic retinopathy (no DR, n=4), and patients with diabetic patients with PDR (n=3). Experienced ophthalmologists diagnosed diabetic retinopathy. Samples were separated by 12% SDS-PAGE and gels were stained with Coomassie™ brilliant blue stain. The entire lane for each sample was then divided equally into 60-70 slices of about 1 mm in width. These individual gel slices were then individually digested with trypsin (Promega, Madison, Wis.) and analyzed by capillary liquid chromatography, nanospray ionization, and tandem mass spectroscopy using LTQ 2-dimensional linear ion trap mass spectrometer (Thermo Electron Corporation). Data acquisition parameters were full scan MS (range 400 to 1200 m/z) followed by 10 data-dependent MS/MS events.

Assignment of MS/MS data was performed using human subset of non-redundant protein database from National Center for Biotechnology Information and TurboSEQUESR (BioWorks™ 3.1, Thermo Electron Corporation). Resultant matches were entered and compiled into the MySQL relational database and proteomics computational analyses were performed using the Hypertext Preprocessor-based interface according to the following algorithm: Peptide identifications were made based on the following criteria: Cross-correlation score >1.5, 2.0 and 2.5 for charge states +1, +2 and +3, respectively; Delta Correlation >0.1; Primary Score >200; Ranking of the Primary Score <3; and percent fragment ions >30%. Protein identifications were assigned when the following criteria were met: unique peptide match number ≧2, peptides contributing to protein matches were derived from a single gel slice or adjacent slices, and the protein was identified in at least 2 vitreous samples. The lower limit of detection was approximately 0.5 to 1 nM.

Protein matches for the 12 vitreous samples were compiled, and the numbers of unique peptides (median and mean±SD) for each protein from the 3 groups of subject are shown in Table 2. The total number of proteins identified in the vitreous was 117, including 64, 113, and 107 proteins in the NDM, noDR, and PDR samples, respectively.

A semi-quantitative comparison of proteins identified in the three groups of vitreous samples (NDM, noDR, and PDR) was performed using the numbers of unique peptides identified by tandem mass spectroscopy for each protein and sample. Kiruskal-Wallis analysis of ranks was used to identify differences (P<0.05) in protein appearance among the three groups.

Kruskal-Wallis analysis of ranks indicated that Dickkopf related protein-3 was detected more frequently in NDM vitreous compared with PDR vitreous.

TABLE 2
DKK3 Differentially Detected in NDM, noDR, and PDR Subjects.

| | | | | | | | | | | Prolif. | | |
|---|---|---|---|---|---|---|---|---|---|---|---|---|
| | | | | | | | | | | Diabet. | | |
| | | | | | | | | | | Retinop. | | |
| | No Diabetes (NDM) | | | | | Diabetes (DM) | | | | (PDR) | | |
| Subject | 1 | 2 | 3 | 4 | 5 | 6 | 7 | 8 | 9 | 10 | 11 | 12 |
| Number | | | | | | | | | | | | |
| No. | 2 | 4 | 5 | 3 | 4 | 6 | 4 | nd | nd | 3 | nd | Nd |
| Frequency | | | | | | | | | | | | |

nd = not detectable

### Example 2

### Characterization of DKK3 Expression in Cells of the Eye

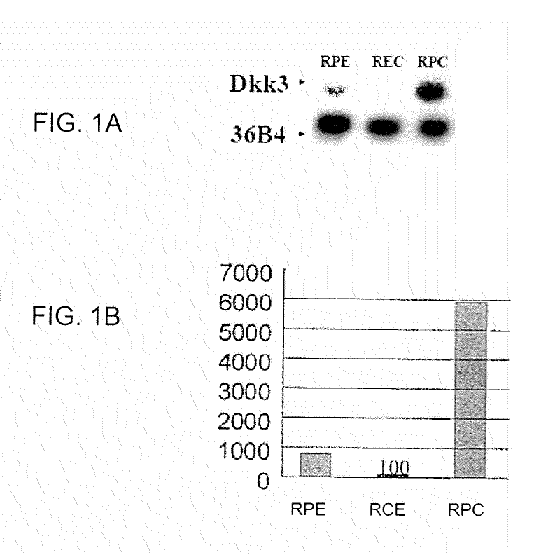

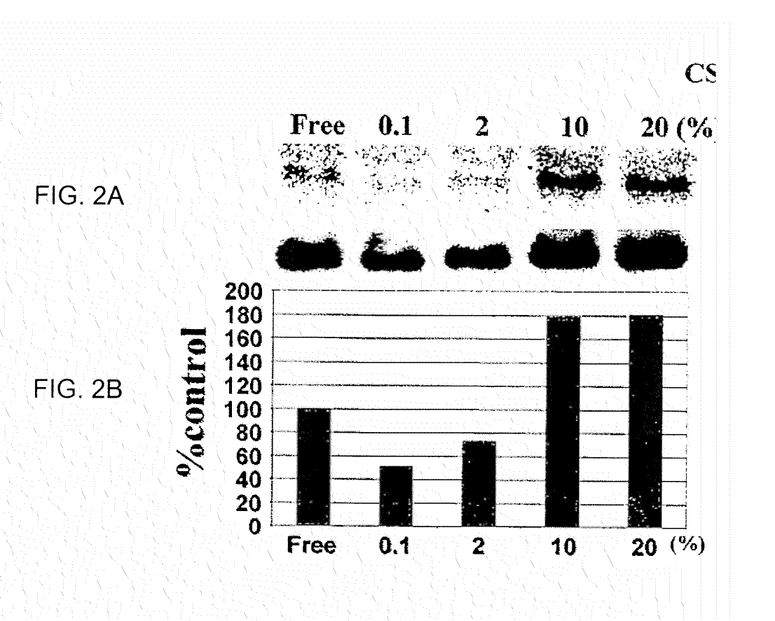

Levels of DKK3 mRNA in murine retinal pericytes (RPs), retinal pigment cells (RPCs), and retinal endothelial cells (RECs) were evaluated using standard Northern blot analysis and quantified using standard methods. RPEs were grown in 10% serum; RPCs, in 20% serum; and RECs in EGM. 10 μg Total RNA was used for analysis. The results, shown in FIG. 1, indicate that DKK3 is mainly expressed in retinal pericytes and retinal pigment cells. It is faint but present in retinal endothelial cells.

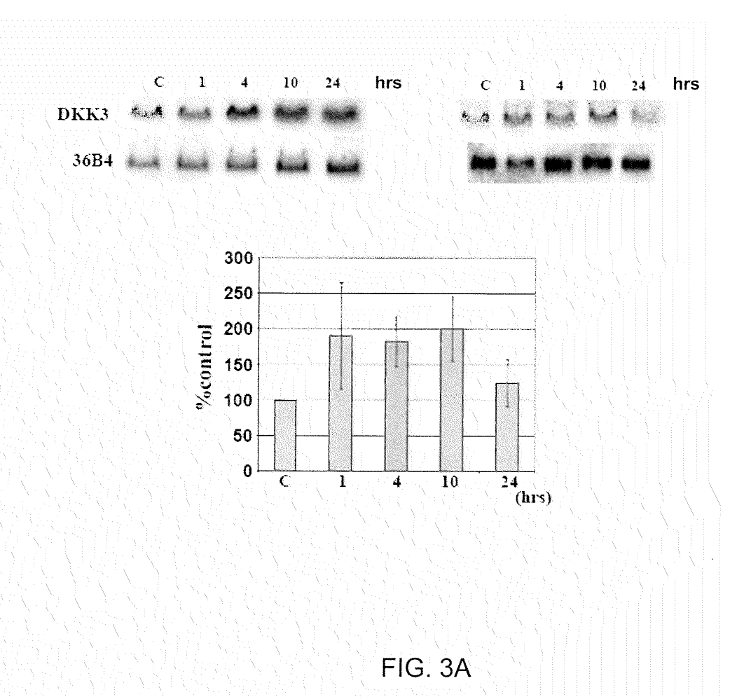

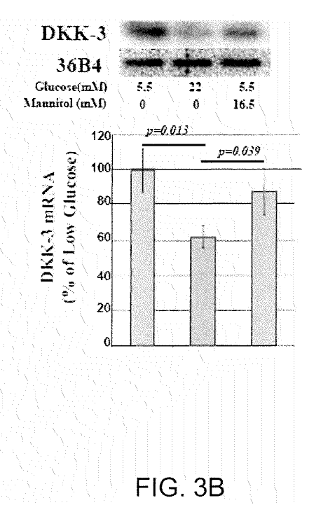

The effect of serum on DKK3 gene expression in RPCs was also evaluated by varying the concentrations of serum in the media in which the RPCs were cultured. Cells were exposed to serum for 24 hours. 12 μg total RNA was probed by Northern blotting. The results, shown in FIGS. 2A and 2B, indicate an effect of serum on DKK3 expression in the pericytes. A small concentration of calf serum seemed to prevent the DKK3 gene expression but 10% and 20% serum stimulate DKK3 expression in pericytes.

The effect of insulin on DKK3 gene expression in RPCs was also evaluated using a time course experiment. Briefly, RPCs (at passage three) were incubated in 2% serum overnight. Cells were then cultured in serum free medium for 8 hours. Insulin was then added at a final concentration of 100 nM and cells were incubated for up to 24 hours. Samples were collected at 0, 1, 4, 10, and 24 hour time points.

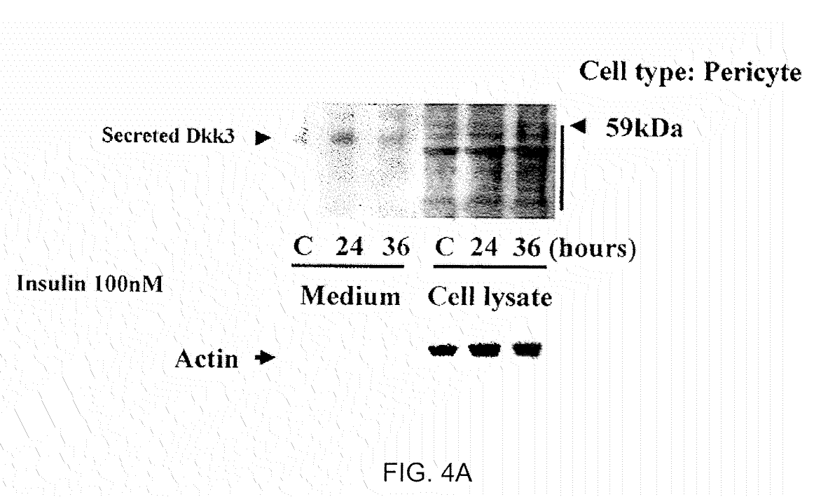

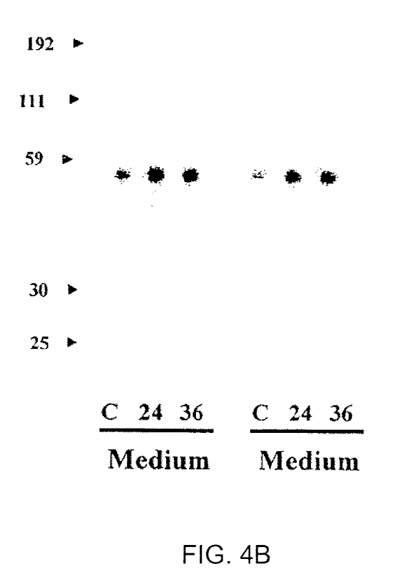

The results, shown in FIG. 3A, demonstrate that insulin induces DKK3 gene expression in pericytes from 1 hour after stimulation. Up to 10 hours after stimulation, DKK3 is still up-regulated compared to control.

The effect of high glucose concentration in RPCs was evaluated. Briefly, cells were cultured for five days in media containing glucose or mannitol (which was included as an osmotic control). Glucose and mannitol concentrations are shown in the top panel of FIG. 3B. As seen in FIG. 3B, high glucose conditions decreased DKK3 gene expression in pericytes.

### Example 3

### DKK3 Protein Expression

DKK3 protein expression was also evaluated using Western blotting, ELISA, and immunoprecipitation using antibodies obtained from R&D (Antigen: whole rhDKK3 (not peptide), manufacturer indicates binds to human (100%) and mouse (35%)) and from Santa Cruz (Antigen: C-end peptides; manufacturer indicates binds to human, mouse, and rat).

Briefly, cells were exposed to 2% serum for 24 hours. Cells were then lysed using Laemmli sample buffer and Western blots were performed using of 8.5 μg of pericyte cell lysate. Prior to probing, membranes were blocked in 1% skim milk for 30 minutes. Cells were then probed with the R&D antibody (described above) at a concentration of 1:100 at room temperature for two hours, followed by a rinse in 1% skimmed milk and incubation with a secondary antibody (Zymed) at a concentration of 1:5000 at room temperature for 30 minutes.

In lysate from HEK 293 cells, heterogeneous bands between 45-65 kDa were seen. Deglycosylation reduced the protein to 40- and 45- to 55-kD proteins. In bovine retinal pericytes, there are four main bands, and incubation in 2% serum could increase DKK3 protein in cell lysates.

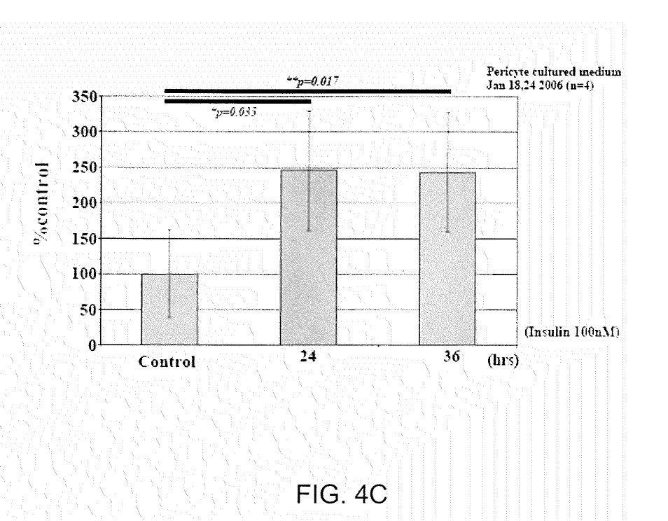

The effect of insulin on DKK3 protein expression was also assessed using Western blotting. Briefly, pericyte cells were exposed to 2% serum for 24 hours and serum free media over night in the presence of 100 mM insulin. Cells were then isolated at 24 and 36 hour time points. Blotting conditions were as described above. 14 μg of cell lysate was loaded per lane. As shown in FIGS. 4A-4C, treatment with insulin increased levels of secreted DKK3.

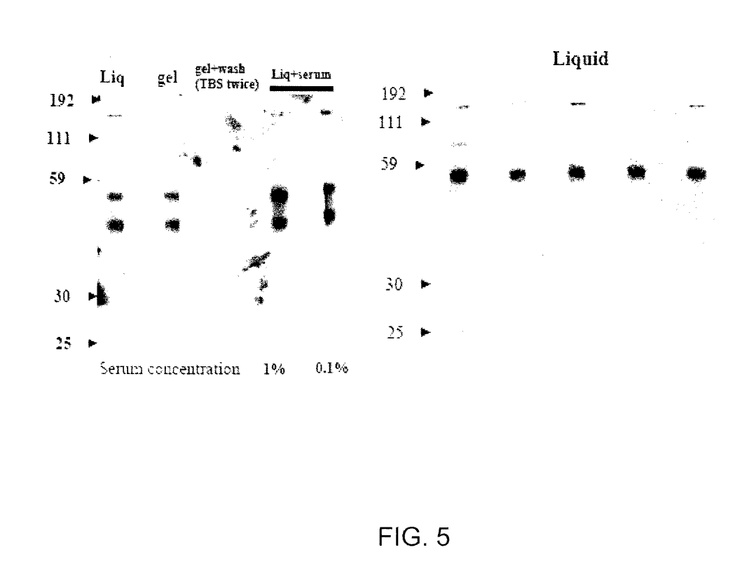

Expression of DKK3 in vitreous was also examined. DKK3 expression was detected using Western blotting. The following samples were obtained from bovine and rat tissues. Liquid vitreous (Liq) and gel-like vitreous (gel) were isolated in lysate buffer and subjected to three rounds of sonication each for a period of 30 seconds. Samples were then centrifuged at 14000 rpm for 2 minutes and the supernatants were collected. A gel-like vitreous sample was also washed twice in TBS prior to lysis and sonication (gel+wash). DKK3 was diluted in this sample, resulting in lower DKK3 detection. The effect of serum on DKK3 expression in the vitreous was examined using serum concentrations of 1% and 0.1% (liquid+serum). Serum was added in a volume of 2 μL into the liquid vitreous (2 μL). Western blots were then performed using 2 μL of cell extract loaded in 8 μL sample buffer. Membranes were probed using a Biotin-conjugated first antibody at a concentration of 1:200 and a second streptoavidin-conjuated horse raddish peroxidase antibody at a concentration of 40 ng/mL. In diabetes, there could be many blood proteins contaminating patient vitreous samples, so the effect of serum on DKK3 protein expression detection was evaluated. The last (left-most) two lanes show the effect of serum contamination on DKK3 protein. The indicated amount of serum (2 microL) was added into the 2 μL liquid vitreous. Together, the results shown in FIG. 5 demonstrate that serum enhances DKK3 protein expression in the vitreous.

### Example 4

### Localization of DKK3 Expression in Mouse Eye and Heart

Previous reports have shown that DKK3 is primarily expressed in forebrain, eye tissues, and heart especially around an atrium. In the eye, DKK3 is expressed in cornea, cilially body, lens and retinas (Gene 238(2):301-13 (1999)).

DKK3 expression was evaluated in mouse cornea.

Staining Procedure

1. Frozen section of Adult mice eye

2. Acetone fix 10 minutes

3. 15 minutes dry

4. PBS washing 15 minutes

5. Blocking by BBS buffer with Rabbit serum 30 minutes R_{T}

6. R&D DKK3 (1:10) in PBS buffer with Rabbit serum O/NR_{T}

7. PBS washing 30 min

8. 2nd anti goat IgG antibody (1:500) 2 hours

9. PBS washing 30 min

10. Mounting

These experiments revealed that DKK3 is mainly expressed around the limbus of cornea, and is detectable in the stroma. In the retina, DKK3 is mainly expressed in the outer nuclear layer and outer plexiform layer.

Expression in rat hearts was also evaluated. The results indicated that DKK3 is expressed in smooth muscle cells, generally outside the vascular endothelial cells.

### Example 5

### DKK-3 in Vitreous of Subjects with PDR

The presence of DKK3 in subjects with PDR was evaluated. The results are shown in Table 3.

TABLE 3
DKK3 expression

| | A | B | C |
|---|---|---|---|
| Vitreous hemorrhage | (+) | (−) | (+) |
| Macular edema | (+) | (+) | (+) |
| Pre-ope Visual Acuity | HM | CF | 20/40 |
| | | | (0.5) |
| Pre-ope Photocoagulation | (+) | (+) | (+) |
| Age/Gender | 57/M | 40/M | 38/M |
| Retinal Detachment | (+) | (+) | (+) |

DKK3 mRNA was detected only in Patient A, and only at low levels. Patient

A had a vitreous hemorrhage. Patient B did not have any vitreous hemorrhage. Patient C also had vitreous hemorrhage, but from the visual acuity, the hemorrhage might not be severe, as there was little visual disturbance. Thus, DKK3 might be associated with vitreous hemorrhage.

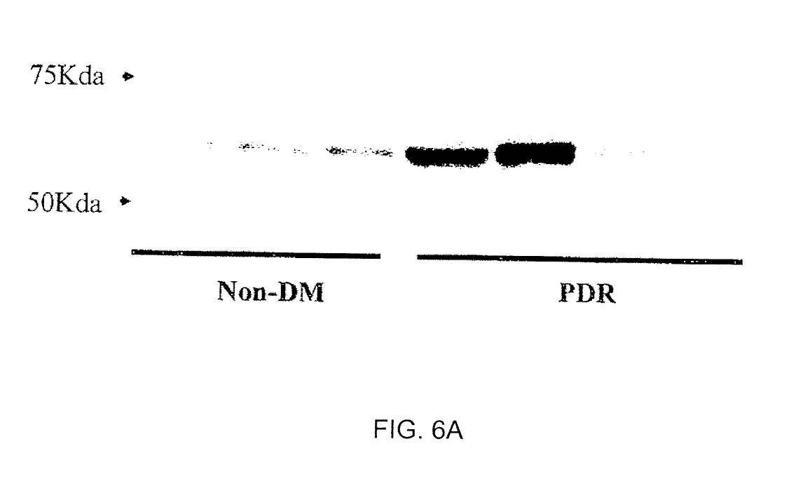

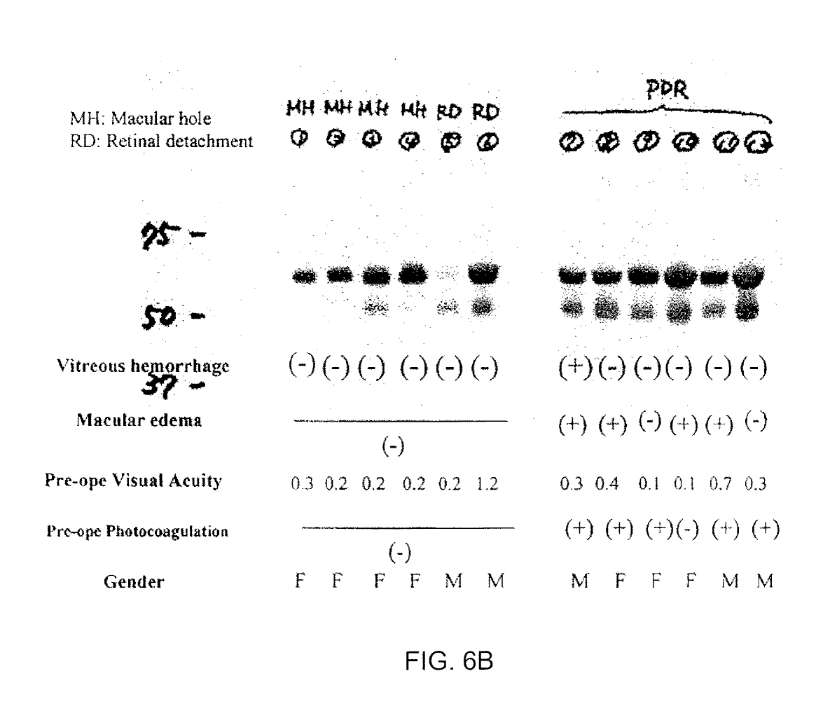

Western Blot results in patient vitreous were inconsistent; in some cases, DKK3 protein appeared to be increased in patients with vitreous hemorrhage (see FIGS. 6A-6B).

### Example 6

### DKK-3 in STZ-Induced Rodent Model of Diabetes

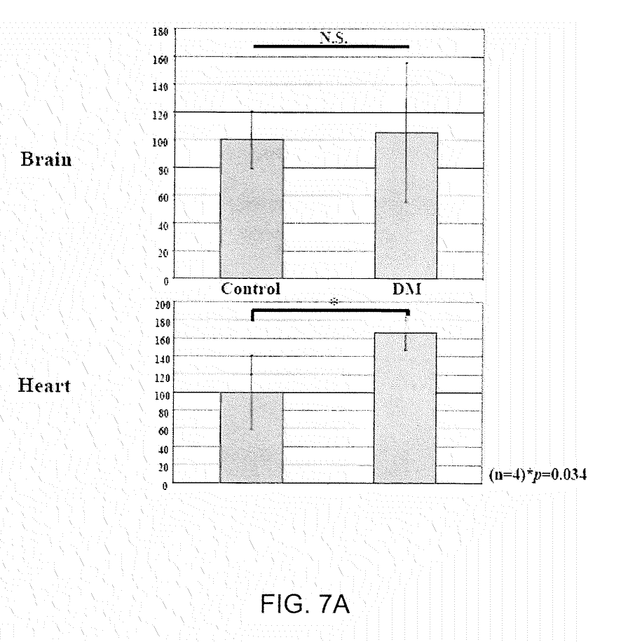

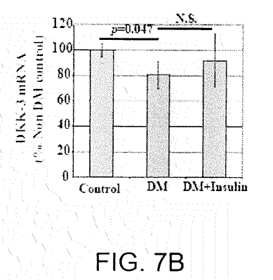

Expression of DKK3 was also evaluated in streptozotocin (STZ)-induced rat model of diabetes. Two months after treatment with STZ, increased DKK3 was seen in rat brain and hearts, and decreased DKK3 was seen in STZ-treated rat retina (see FIGS. 7A-7B).

### Example 7

### DKK-3 in Vitreous of Subjects with PDR

DKK3 has previously been reported to be a tumor suppressor. Thus, the effect of DKK3 on cell proliferation was evaluated in RPEs and Aortic endothelial cells (AoEC). RNAi was used to decrease expression levels to model reduced levels of DKK3 seen in DR retina.

Two DKK3 siRNA sequence were designed using the Dharmacon RNAi design tool. Both sequences were directed to a region of the sequence that is conserved between human and cow DKK3. Both sequences were effective, as evaluated by real-time PCR and western blotting in both human and bovine cultured cells. The first DKK3 siRNA sequence for bovine and human cultured cell is from nt 866-885:

| | | |
|---|---|---|
| sense: | AGAUGUUCCGCGAGGUUGAdTdT | (SEQ ID NO: 1) |
| antisense: | UCAACCUCGCGGAACAUCUdTdT | (SEQ ID NO: 2) |

The second DKK3 siRNA sequence for bovine and human is from nt 1056-1075:

| | | |
|---|---|---|
| sense: | ACCGAGAAAUUCACAAGAUdTdT, | (SEQ ID NO: 3) |
| antisense: | AUCUUGUGAAUUUCUCGGUdTdT | (SEQ ID NO: 4) |

The latter was used for BoAoECs migration assays.

The protocol used was as follows:

Bovine AoECs (Passage 7) and BRPE (Passage 1) were used. DKK3/GFP RNAi (final concentration of 100 nM), were transfected using Oligofectamin (6 μL/1.2 mL 10% serum+DMEM). Seventy two hours after transfection, RNA isolation was performed using RNeasy Kit with RT: superscript III (0.2 μg). Forty cycles of Real time PCR (RT-PCR) was performed. Seven days after transfection, protein samples were isolated and evaluated by western blotting.

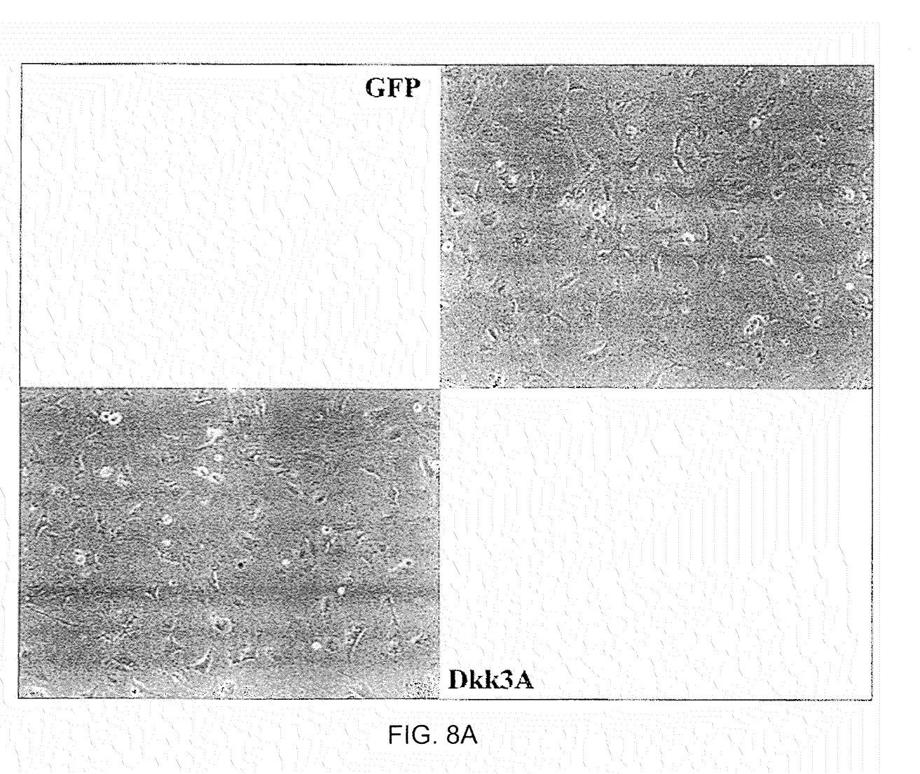

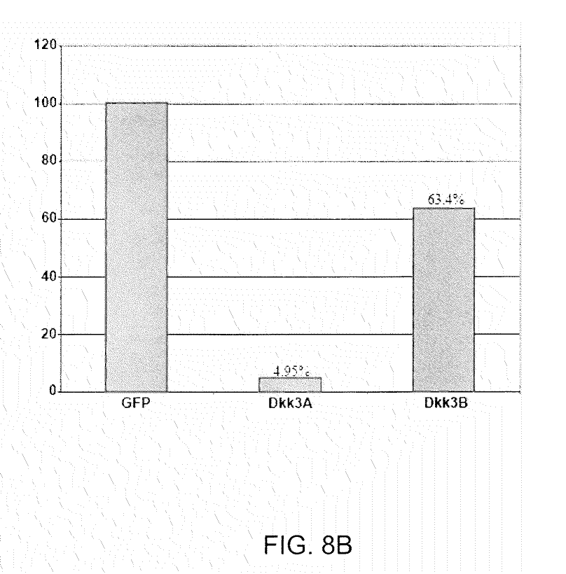

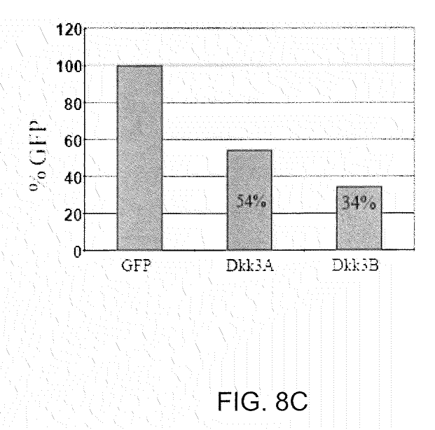

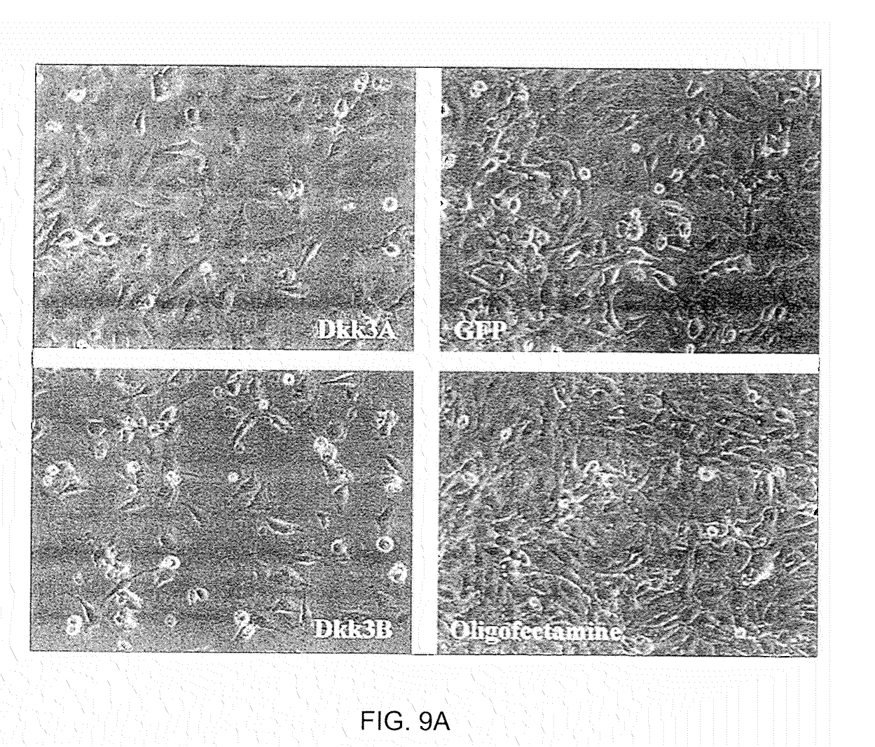

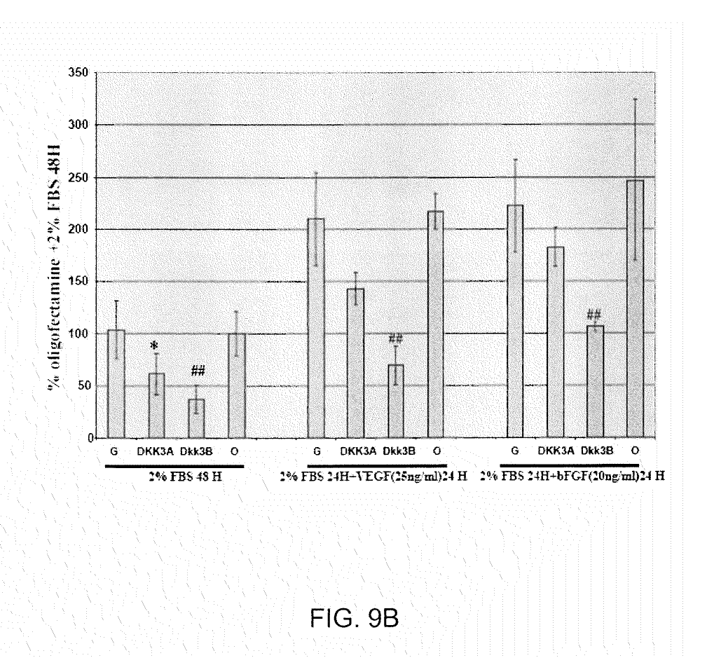

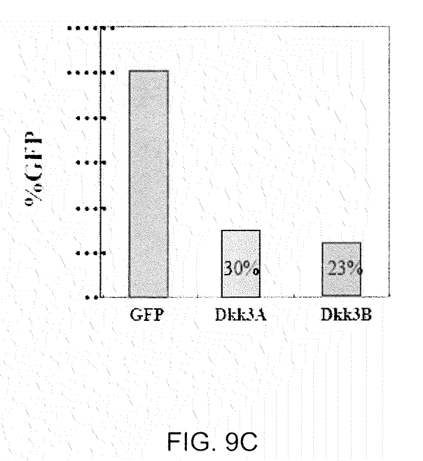

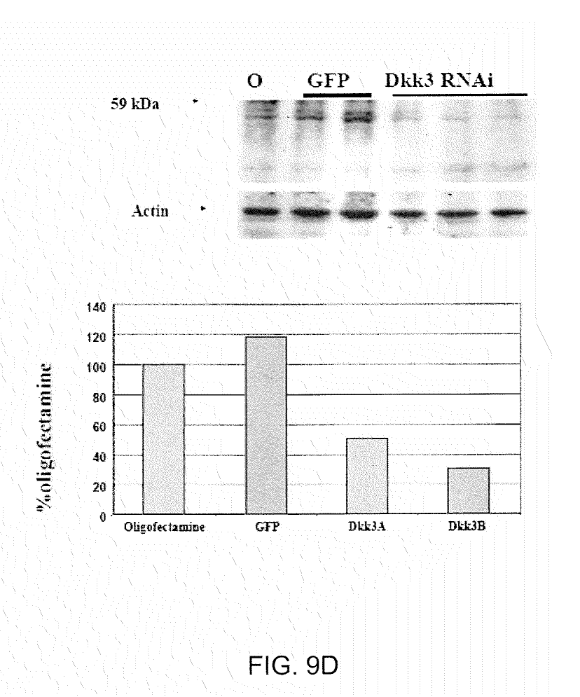

In the RPEs, DKK3 RNAi resulted in a decrease in DKK3 mRNA (FIGS. 8B-8C), and a small decrease in confluence when measured two days after transfection (FIG. 8A). Similar results were seen in the bovine AoECs (FIGS. 9A and 9C), and were confirmed by an assay to determine cell numbers (FIG. 9B) and Western blot (FIG. 9D).

### Example 8

### Expression of DKK Family Members in Human Vitreous

Expression of DKK family members in human vitreous was examined in 12 subjects (No DM=5, DM/no DR=4, PDR=3). The results showed no detectable expression of DKK1, DKK2, or DKK4 in any of the subjects, while DKK3 was detected in 8/12. PEDF, used as a control, was detected in 12/12.

### Example 9

### Effects of Recombinant DKK-3 on Cell Proliferation and Signalling

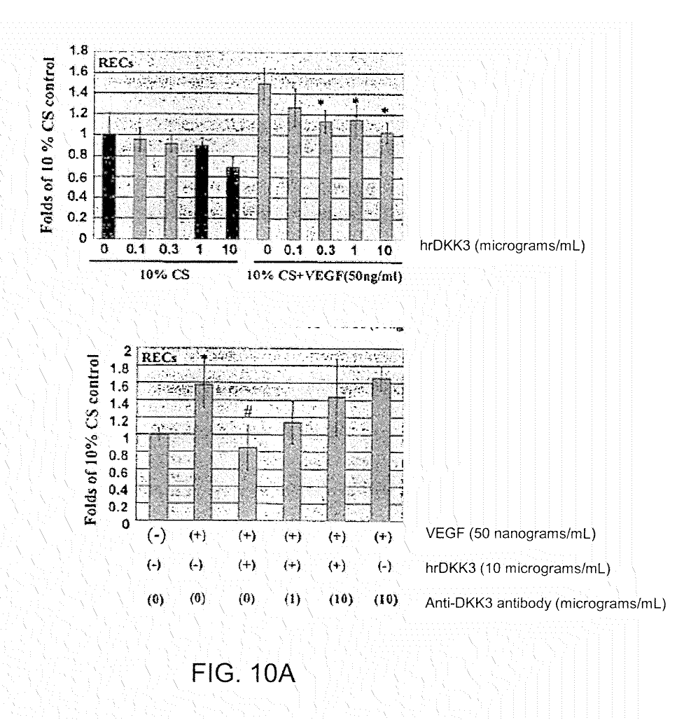

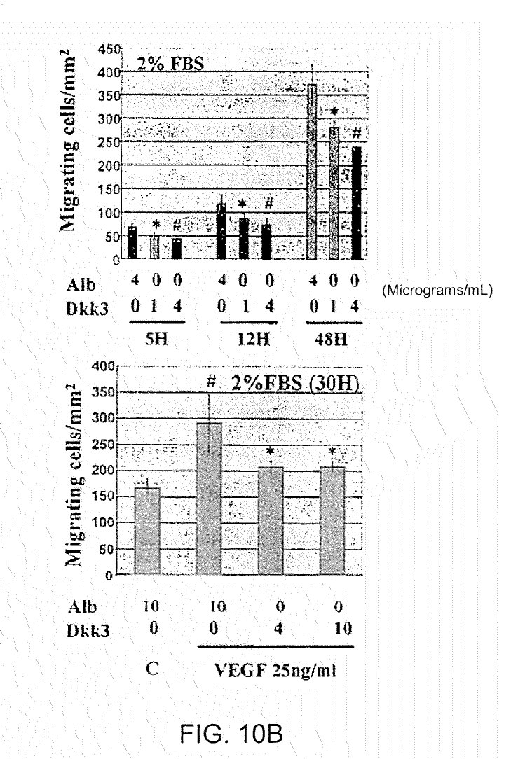

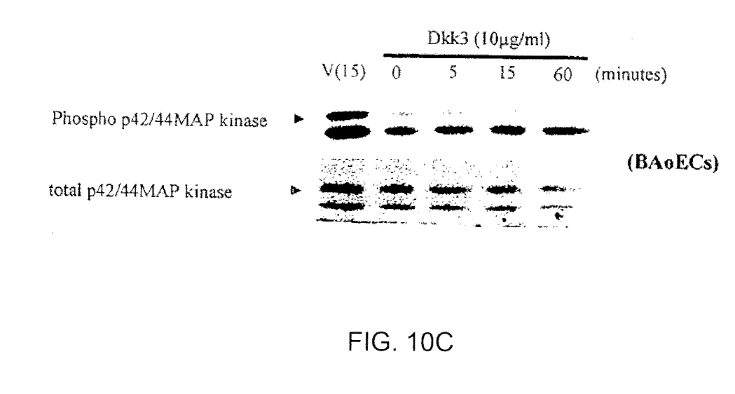

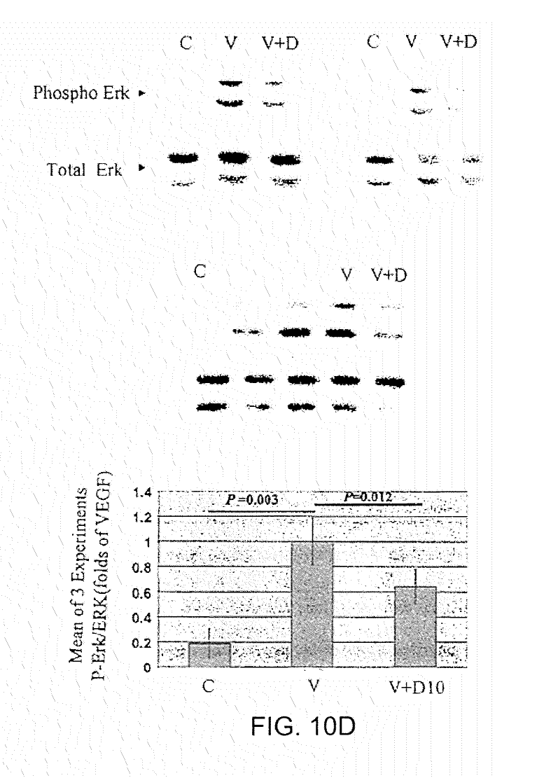

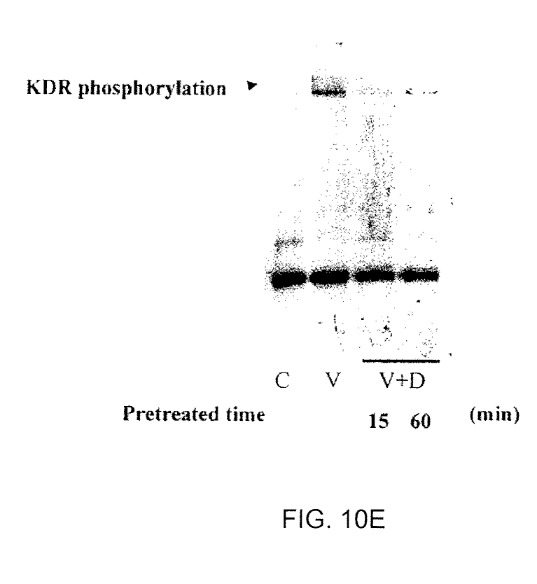

Human recombinant DKK3 was obtained from R&D systems (Minneapolis, Minn.) and applied exogenously to RECs. Briefly, cells were treated with or without VEGF as a stimulant of retinal endothelial cell growth. The effect of DKK3 on VEGF-dependent retinal endothelial cell growth was then determined using BrdU. Proliferation in the presence and absence of VEGF stimulation was then assayed. The results, shown in FIG. 10A, demonstrate that DKK3 was able to completely inhibit VEGF-induced REC proliferation.

Migration of RECs was also delayed, in the presence and absence of VEGF (FIG. 10B). The results demonstrated that DKK3 delayed endothelial cell migration, with maximal effect at 4 μg/ml.

In addition, DKK3 did not enhance ERK phosphorylation, but did inhibit VEGF-induced ERK phosphorylation as well as KDR phosphorylation (FIGS. 10C-E).

To evaluate whether DKK3 was interacting directly or indirectly with VEGF or KDR, a functional ELISA and co-immunoprecipitation assay were carried out.

Briefly, wells in a 96-well plate were coated with 1000 ng/mL DKK3 or 1000 ng/mL albumin overnight at 4° C. Wells were then blocked using a phosphate buffered saline solution containing 1% albumin and 0.1% tween. Recombinant VEGF was then added to the appropriate wells. Following extensive washing, VEGF was detected using anti-VEGF antibody diluted in PBS. Bound antibody was detected using anti-mouse IgG antibody diluted in PBS. All antibody incubation steps were performed at 37° C. Multiple wash steps were performed following each antibody incubation.

Alternatively, 3 μg Anti-DKK3 antibody was immobilized on 10 μL Protein AG-agarose beads per sample. Beads were then washed three times in washing buffer to remove unbound antibody. 1.25 μg of DKK3 or albumin was then added per sample in immunoprecipitation buffer and samples were incubated overnight at 4° C. Samples were then washed twice in washing buffer and once in 1× binding buffer (20 mM HEPES pH 7.3; 150 mM NaCl; 0.5% NP40; 1% bovine serum albumin (BSA); 1 mM CaCl₂; and 1 mM MgCl₂). Binding reactions were performed in 1 mL 1× binding buffer. Briefly, I-VEGF (1 μL, 80000 cpm) with and without cold VEGF (100 ng/mL) was incubated per sample for 2 hours at 4° C. Samples were then washed four times in washing buffer to removed unbound VEGF. Bound VEGF was detected by scintillation.

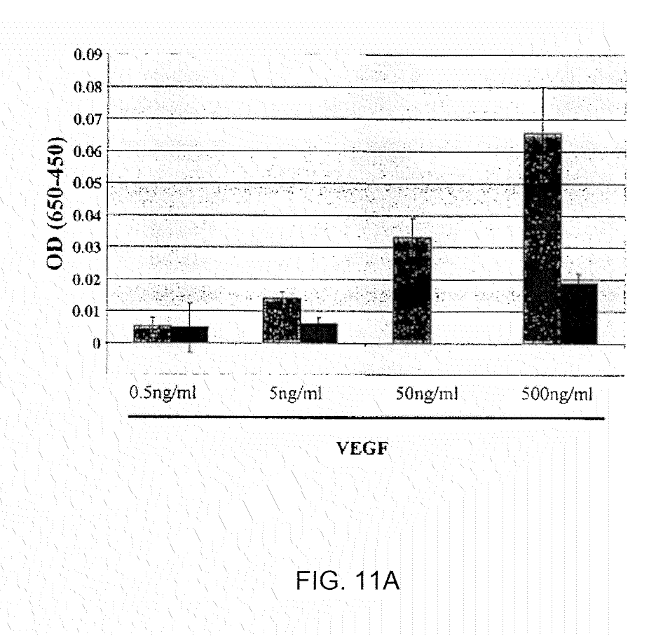

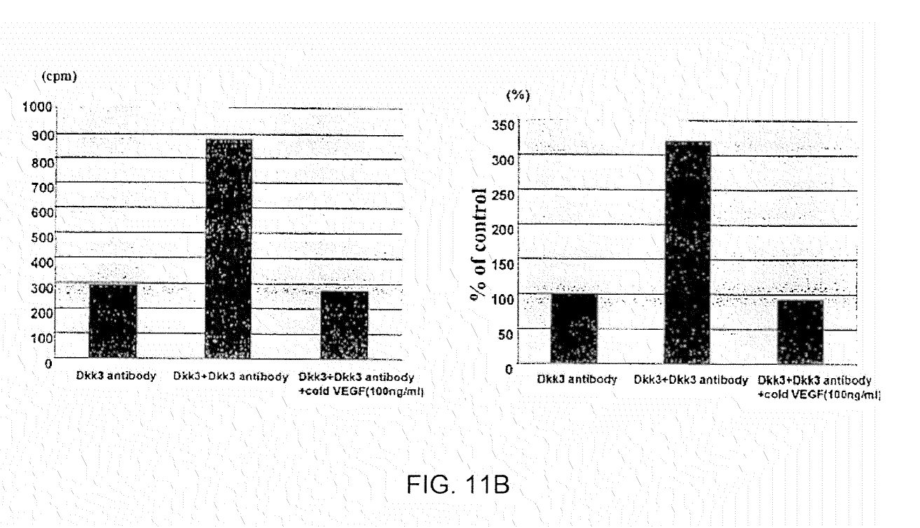

As shown in FIGS. 11A-B, DKK3 showed interaction with rhVEGF in a dose dependent matter.

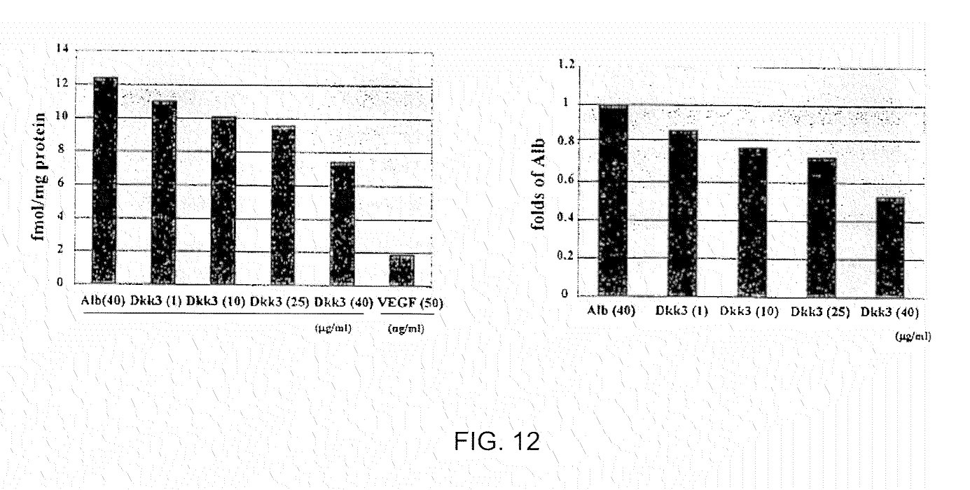

Next, the effect of DKK3 on VEGF-VEGFR interactions was evaluated. RECs were seeded on 12 well plates and grown to confluence. The cells were washed with cold washing buffer (0.1% alb) and the dishes were placed on ice. Binding buffer was added, with either cold VEGF or rhDKK3. I-VEGF was added (50000 cpm/each well). Cells were incubated at 4° C. for 4 hours, then washed with washing buffer. Proteins were isolated in 0.1% SDS and binding evaluated by scintillation counter. Results were normalized by protein concentration. As shown in FIG. 12, DKK3 has a slight but clear inhibitory effect on VEGF-VEGF receptor binding, about 25% at 10 μg/ml. At 40 mg/ml DKK3 inhibits 50% of VEGF-VEGF receptor binding.

### Example 10

### Effect of Recombinant DKK-3 on Retinal Vascular Permeability

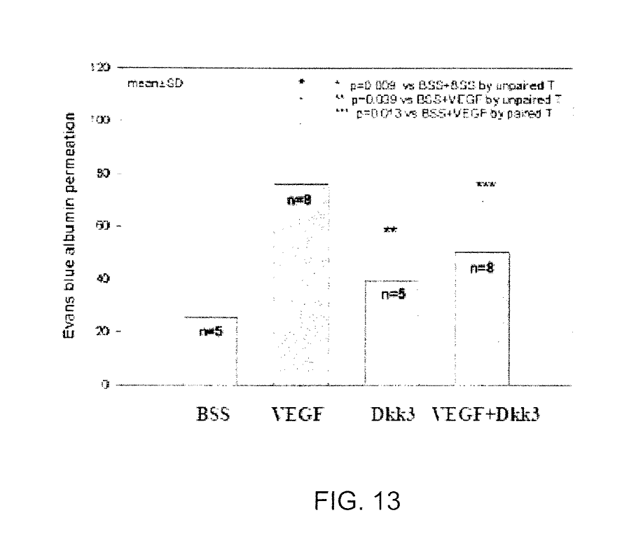

Evans blue albumin permeability assay was used to detect the effect of recombinant DKK3 on VEGF-induced vascular permeability. Assays were conducted as described in International Patent Application No. PCT/US2006/005395 (publication No. WO 2006/091459), filed on Feb. 16, 2006, which relates to compounds and methods for diagnosing and treating disorders associated with excessive vascular permeability and edema in the retina and brain The results, shown in FIG. 13, demonstrated that DKK3 itself has a slight inducible effect on vascular permeability but clearly inhibited VEGF induced retinal permeability.

### Example 11

### Effect of Recombinant DKK-3 on Endothelial Cell Viability

The effect of DKK3 on endothelial cell viability was evaluated in RECs and HUVECs. An MTT assay was used to evaluate cell viability; this assay evaluates cell mitochondrial activity. MTT, a yellow liquid, is incorporated by living cells and metabolized in the mitochondria, which changes its color to violet. That color change was evaluated with a microreader.

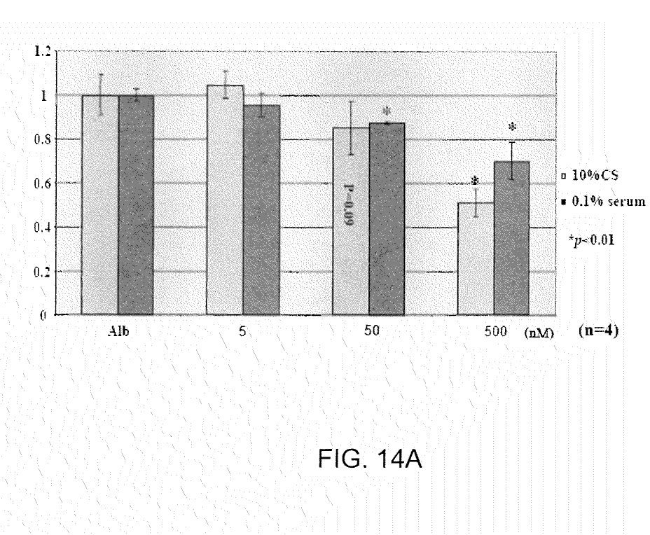

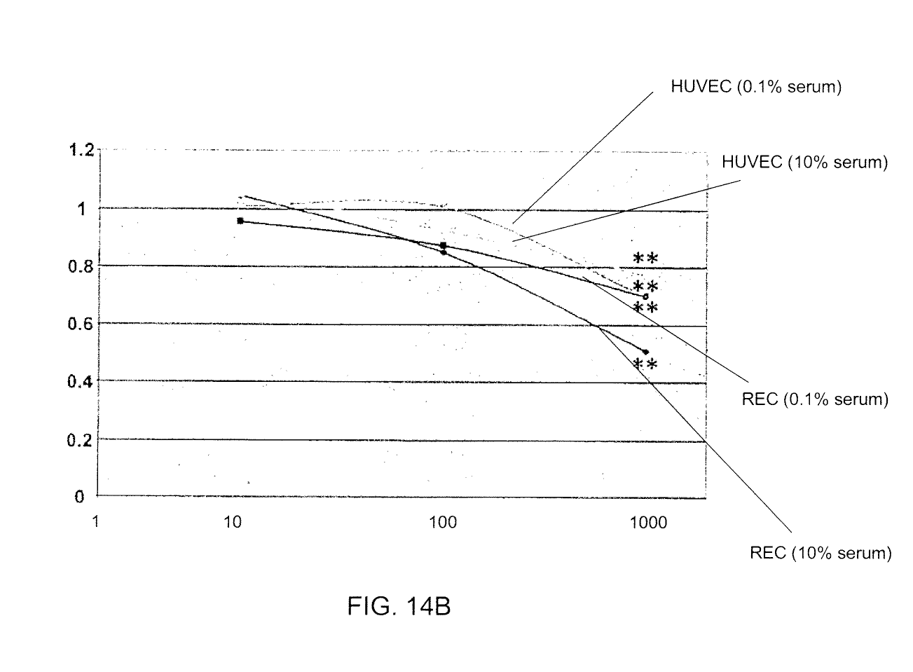

In both 0.1% and 10% CS conditions, the cell number decreased in DKK3 condition. As shown in FIGS. 14A-14B, DKK3 reduced cell viability.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of decreasing retinal vascularisation or vascular permeability in the eye of a subject, the method comprising administering to the subject a therapeutically effective amount of a composition that increases activity or levels of a Dickkopf 3 (DKK3) polypeptide in the subject.

2. The method of claim 1, wherein the composition comprises a DKK3 polypeptide or active fragment thereof, or a nucleic acid encoding DKK3 or an active fragment thereof.

3. The method of claim 1, wherein the composition is administered locally to the eye of the subject.

4. The method of claim 3, wherein the local administration is by injection into the vitreous or aqueous humor of the eye.

5. The method of claim 3, wherein the composition is administered as eye drops.

6. The method of claim 1, wherein the subject has diabetic retinopathy.

7. A pharmaceutical composition comprising a compound that increases activity or levels of DKK3 and a physiologically acceptable carrier.

8. The composition of claim 7, wherein the compound comprises a DKK3 polypeptide or active fragment thereof, or a nucleic acid encoding DKK3 or an active fragment thereof.

9. The composition of claim 7, wherein the composition is adapted for injection into the vitreous or aqueous humor of a mammalian eye, by providing the composition in a form that is sterile, substantially free of endotoxin, and within a physiologically acceptable pH.

10. The composition of claim 7, wherein the composition is adapted for use as eye drops.

11. A method of identifying a candidate compound for the treatment of a disorder associated with excessive vascularisation or vascular permeability of the eye, the method comprising:
assaying DKK3 levels or activity in the presence of a test compound; and
identifying a test compound that increases DKK3 levels or activity, relative to a reference,
wherein the test compound that increases DKK3 levels or activity, relative to a reference, is a candidate compound for the treatment of the disorder.

12. A method of identifying a candidate compound for the treatment of a disorder associated with excessive retinal vascularisation or vascular permeability in the eye, the method comprising:
administering a test compound to an animal;
detecting a level of DKK3 protein or mRNA encoding DKK3 in the animal or a sample from the animal; and
comparing the level of the DKK3 protein or mRNA to a reference,
wherein a test compound that causes an increase in a level of the DKK3 protein or mRNA as compared to the reference is a candidate compound for the treatment of a disorder associated with excessive retinal vascularisation or vascular permeability.

13. The method of claim 12, wherein the animal is an animal model for the disorder.

14. A method of diagnosing a subject with a disorder associated with excessive retinal vascular permeability of the eye, the method comprising:
detecting a level of DKK3 protein or mRNA encoding DKK3 in a sample from the subject; and
comparing the level of the DKK3 protein or mRNA to a reference,
wherein a decrease in a level of the DKK3 protein or mRNA as compared to the reference indicates that the subject has a disorder associated with excessive retinal vascularisation or vascular permeability.

15. A method of evaluating a treatment for a disorder associated with excessive vascular permeability, the method comprising:
detecting a level of DKK3 protein or mRNA encoding DKK3 in a sample from the subject;
administering one or more doses of a treatment, and
comparing the level of the DKK3 protein or mRNA encoding DKK3 to a reference,
wherein a difference in a level of the DKK3 protein or mRNA, as compared to the reference, indicates the efficacy of the treatment.

16. The method of claim 15, wherein the reference represents a level of the protein or mRNA prior to administration of the treatment.

17. The method of claim 15, wherein the sample is from the eye of the subject.

18. A method of determining a subject's risk for development of a sight-threatening complication of retinopathy, the method comprising:
detecting a level of DKK3 protein or mRNA encoding DKK3 in a sample from the subject; and
comparing the level of the DKK3 protein or mRNA to a reference,
wherein a difference in a level of the DKK3 protein or mRNA as compared to the reference indicates the subject's risk of developing a sight-threatening complication of retinopathy.

19. A method of determining when a treatment modality administered to a subject to treat a disorder associated with excessive retinal vascularisation or vascular permeability can be stopped, the method comprising:
detecting a level of DKK3 protein or mRNA encoding DKK3 in a sample from the subject;
comparing the level of the DKK3 protein or mRNA to a reference,
wherein a level of the DKK3 protein or mRNA that approaches the level of the protein or mRNA in the reference indicates whether the treatment can be stopped.

20. A method of determining when a treatment for a disorder associated with excessive vascular permeability should be initiated in a subject, the method comprising:
detecting a level of DKK3 protein or mRNA encoding DKK3 in a sample from the subject; and
comparing the level of the DKK3 protein or mRNA to a reference,
wherein a significant difference in a level of the DKK3 protein or mRNA as compared to the reference indicates whether the treatment should be initiated.

21. The method of claim 20, wherein the sample is from the eye of the subject.

22. The method of claim 20, wherein the reference represents a level of the DKK3 protein or mRNA in an unaffected subject.

23. The method of claim 20, wherein the reference represents a level of the DKK3 protein or mRNA in an affected subject.
